(19) United States
(12) Patent Application Publication
(10) Pub. No.: US 2004/0236309 A1
(43) Pub. Date: Nov. 25, 2004

(54) Mesh ventricular catheter with antithrombogenic coating

(76) Inventor: Benson Yang, Chicago, IL (US)

Correspondence Address: WELSH & KATZ, LTD, 120 S RIVERSIDE PLAZA, 22ND FLOOR, CHICAGO, IL, 60606, US

(21) Appl. No.: 10/440,843

(22) Filed: May 19, 2003

(51) Int. Cl. A61M 25/00

(52) U.S. Cl. 604/523; 604/265

(57) ABSTRACT

A catheter is provided for use within the cerebral ventricle of the human body. The catheter includes a tubing substrate, a semipermeable mesh of a relatively constant sieve size disposed over the substrate and an antithrombogenic coating disposed on all surfaces of the semipermeable mesh.

### FIELD OF THE INVENTION

The field of the invention relates to medical devices, and more particularly, to catheters.

### BACKGROUND OF THE INVENTION

This invention relates to surgically implanted drainage catheters. Such devices are often used as part of shunt systems to divert cerebrospinal fluid from the brain to another part of the body.

Shunt systems have widespread use in the treatment of hydrocephalus. Cerebrospinal fluid is continuously produced in the brain and normally circulated through the central nervous system before being absorbed into the bloodstream. In the case of hydrocephalus, fluid that should drain away, instead, accumulates within the cerebral ventricles thereby causing elevated intracranial pressure. The pressure is transmitted to sensitive brain structures resulting in neurological debilitation or even death.

Alteration of normal cerebrospinal fluid pathways can result from a congenital defect, intraventricular hemorrhage, brain injury, infection, or brain tumor. Hydrocephalus most commonly occurs in children, although adults can be similarly afflicted by the aforementioned etiologic mechanisms.

The treatment of hydrocephalus typically involves the insertion of a ventricular catheter through a burr hole in the skull. The ventricular catheter is connected to a pressure valve and distal tubing that is tunneled subcutaneously to shunt cerebrospinal fluid to another part of the body, most commonly the peritoneal cavity of the abdomen.

While shunt systems described above are life saving, they are also subject to malfunction, most commonly due to obstruction of the ventricular catheter. In such cases, negative pressure within the catheter lumen encourages surrounding tissue to grow into the openings of the catheter thereby blocking the proper functioning of the catheter.

Another common cause of ventricular catheter obstruction is encrustation of catheter openings by proteinaceous or cellular matter. Such is a challenge for all implanted devices since most foreign materials instigate the coagulation cascade and invite protein adhesion.

Some children undergo tens to hundreds of operations for shunt revision. Because of the importance of implantable catheters, a need exists for a shunt system whose operation is not impeded by ingrowing tissue and whose components are biologically passivating.

### SUMMARY

A novel catheter is provided for use within the cerebral ventricles of the human body. The catheter includes an impermeable tubular substrate, a mesh of a relatively constant sieve size disposed over the substrate and an antithrombogenic coating disposed on all surfaces of the mesh.

### BRIEF DESCRIPTION OF THE DRAWINGS

FIG. 1 illustrates the use of a shunt system implanted in a patient 12 for the treatment of hydrocephalus; and

FIG. 2 depicts a detailed view of a ventricular catheter that may be used with the system of FIG. 1.

### DETAILED DESCRIPTION OF AN ILLUSTRATED EMBODIMENT

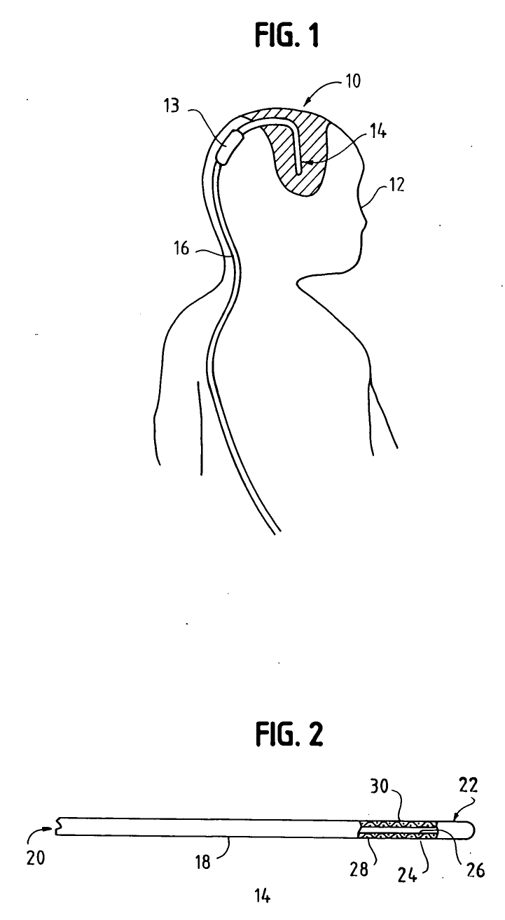

FIG. 1 depicts a shunt system 10 that may be used in the treatment of hydrocephalus. As shown, the system 10 may include a catheter 14 implanted into a ventricle within the cranial cavity of a patient 12. A valve mechanism 13 may be connected to an open end of the ventricular catheter 14 to regulate drainage pressure. A separate tube 16 may be connected to the other end of the valve mechanism 13 to shunt fluid to another part of the body of the patient (e.g. the peritoneal cavity).

FIG. 2 shows a side view of the ventricular catheter 14. As shown, the ventricular catheter 14 may include a semipermeable mesh filter assembly 24 supported by a substrate. The substrate may include a body 18 (e.g., made of silastic elastomer tubing) with a number of slots 26. The body 18 may also include a distal tube connection end 20 and a proximal infusion end 22.

The mesh filter assembly 24 that is disposed on the infusion end 22 allows free passage of fluids into a lumen that extends the length of the body 18, through the valve mechanism 13 and out through the tube 16. Concurrently, the mesh filter assembly 24 is a barrier to the ingrowth of tissue. The unique combination of free diffusion to fluids and impermeability to tissue proliferation allows the ventricular catheter to function more effectively than shunt devices described in the prior art.

The mesh filter assembly 24 may include a mesh filter element 30. The mesh filter element 30 may be constructed in such a way as to provide an array of openings of a relatively constant sieve size. In general, the openings may be selected of a macrocellular size that is capable of allowing single cells or small groups of cells to pass through, but which are too small to allow for tissue ingrowth. As used herein, a square mesh sieve of a macrocellular size is in the range from 50 to 150 microns on each side, with a preferred size of approximately 100 microns on each side.

A factor essential to the long-term functioning of such a ventricular catheter is the use of an antithrombogenic substance 28 to provide a biologically inert coating on the surface of the mesh filter element 30. What had not been recognized in the prior art are the benefits of combining a macrocellular opening size with a passivating substance that reduces cellular and proteinaceous adhesion.

It has been known in the art that most foreign materials trigger the coagulation cascade in the presence of blood. This phenomenon is exaggerated for semipermeable barriers with small pores since the ratio of reactive surface area to drainage area is high. As a result, semipermeable barriers by themselves have not been successfully employed in implanted catheters. Current catheters are generally constructed with large apertures, with diameters of 500 microns or greater, directly bored into the tubular substrate.

The mesh filter assembly 24 may be created under any of a number of processes. For example, a mesh filter element 30 comprised of a metal (e.g., stainless steel) or polymer (e.g., polyethylene) is provided with a relatively constant sieve size (e.g., approximately 100 microns on a side). The mesh surface may then be coated with an antithrombogenic coating 28 (e.g. polyvinylpyrrolidone and/or heparin).

The catheter 14 may be prepared by creating two or more slots 26 disposed around the infusion end 22 of the substrate 18 (e.g., silastic elastomer tubing) with the longitudinal axis of each slot 26 aligned parallel with the length of the substrate 18. The end of the substrate 18 is tapered beyond the mesh filter element 30 to a blunt tip that is minimally destructive to brain tissue during catheter implantation.

A biocompatible adhesive (e.g., silicone-based) may be disposed around an outside surface of the infusion end 22 of the substrate 18, around the longitudinally arranged slots 26. A single layer of the mesh filter element 30 may be trimmed to size and adhered circumferentially around the infusion end 22 to completely overlie the slots 26.

The presence of the macrocellular sieve allows fluids, protein, and small cellular aggregates to easily pass through the mesh filter assembly 24. The importance of selecting a macrocellular mesh sieve to allow clearance of cellular debris had not been appreciated in the prior art. In addition, the mesh filter assembly 24 prevents brain tissue from proliferating into and obstructing the filter assembly, thereby reducing the possibility of catheter obstruction. The passivating antithrombogenic coating 28 retards any adherence of proteinaceous or cellular components to the mesh filter assembly 24 thereby assuring the catheter 14 a longer useful life than that experienced by prior art catheters.

A specific embodiment of a method and apparatus for providing a catheter has been described for the purpose of illustrating the manner in which the invention is made and used. It should be understood that the implementation of other variations and modifications of the invention and its various aspects will be apparent to one skilled in the art, and that the invention is not limited by the specific embodiments described. Therefore, it is contemplated to cover the present invention and any and all modifications, variations, or equivalents that fall within the true spirit and scope of the basic underlying principles disclosed and claimed herein.

1. A ventricular catheter for use within a ventricle of a human body, such catheter comprising:
a substrate;
a filter assembly of a relatively constant sieve size disposed over the substrate; and
an antithrombogenic coating disposed on all surfaces of the semipermeable mesh.

2. The catheter as in claim 1 wherein the substrate further comprises a tube.

3. The catheter as in claim 2 wherein the tube further comprises a plurality of aperatures disposed in a wall of the tube.

4. The catheter as in claim 3 wherein the plurality of apertures disposed in a wall of the tube further comprises a plurality of longitudinal slots disposed in the tube.

5. The catheter as in claim 1 wherein the filter assembly further comprises a mesh fabric of a relatively constant sieve size.

6. The catheter as in claim 5 wherein the mesh fabric further comprises stainless steel.

7. The catheter as in claim 5 wherein the mesh fabric further comprises polyethylene.

8. The catheter as in claim 5 wherein the mesh fabric further comprises a sieve size selected from a range of sieve sizes that lie between 50 and 150 microns on each side.

9. The catheter as in claim 5 wherein the filter assembly further comprises a single layer of the mesh fabric disposed around the tube.

10. The catheter as in claim 5 wherein the mesh further comprises a nominal pore size of 100 microns.

11. The catheter as in claim 1 wherein the antithrombogenic coating further comprises polyvinylpyrrolidone.

12. The catheter as in claim 1 wherein the antithrombogenic coating further comprises heparin.

13. A catheter for use within a ventricle of a human body, such catheter comprising:
a tube with a plurality of longitudinal slots disposed in the tube;
a mesh fabric disposed around the tube covering the plurality of longitudinal slots; and
a coating of an antithrombogenic substance disposed on a surface of the mesh fabric.

14. The catheter as in claim 13 wherein the mesh further comprises stainless steel.

15. The catheter as in claim 13 wherein the mesh further comprises polyethylene.

16. The catheter as in claim 13 wherein the mesh further comprises a sieve size of from 50 to 150 microns.

17. The catheter as in claim 13 wherein the mesh further comprises a nominal pore size of 100 microns.

18. The catheter as in claim 13 wherein the antithrombogenic substance further comprises polyvinylpyrrolidone.

19. The catheter as in claim 13 wherein the antithrombogenic substance further comprises heparin.

20. The catheter as in claim 13 wherein the mesh fabric further comprises a single layer of the mesh fabric disposed around the tube.

21. A catheter for use within a ventricle of a human body, such catheter comprising:
a tube with a plurality of longitudinal slots disposed in the tube; and
a mesh fabric possessing macrocellular sieves disposed around the tube covering the plurality of longitudinal slots.

22. The catheter as in claim 21 wherein the mesh fabric further comprises a coating of an antithrombogenic substance disposed on a surface of the mesh fabric.

23. The catheter as in claim 21 wherein the mesh further comprises stainless steel.

24. The catheter as in claim 21 wherein the mesh fabric comprises polyethylene.
